(19) United States
(12) Patent Application Publication
(10) Pub. No.: US 2011/0027305 A1
(43) Pub. Date: Feb. 3, 2011

(54) Composition Comprising an Extract of Herbal Combination Thereof for Preventing and Treating Diabetes Mellitus

(75) Inventor: Sung Young Lee, Seoul (KR)

Correspondence Address: THE WEBB LAW FIRM, P.C., 700 KOPPERS BUILDING, 436 SEVENTH AVENUE, PITTSBURGH, PA, 15219, US

(73) Assignee: DAI HAN PHARM. DO., LTD., Seoul (KR)

(21) Appl. No.: 12/848,568

(22) Filed: Aug. 2, 2010

(30) Foreign Application Priority Data
Aug. 3, 2009 (KR) 10-2009-0071210

(51) Int. Cl. A61K 36/068; A61P 3/10; A61P 9/12; A61P 9/10; A61P 13/12; A61P 25/02

(52) U.S. Cl. 424/195.15

(57) ABSTRACT

Provided is a composition for treating and preventing diabetes or diabetic complications comprising an extract of herbal combination as an effective ingredient. The composition for treating and preventing diabetes or diabetic complications includes an extract of herbal combination of bitter melon, caterpillar fungus, wolfberry tree bark, white mulberry bark, winged euonymus, kudzu root, Solomon's seal, white atractylodes rhizome, mondo grass rhizome, Japanese cornel fruit and ginseng as an effective ingredient. The composition increases secretion of insulin in a type 2 diabetic animal, improves insulin resistance and exhibits potent blood sugar-lowering and diabetic complication inhibiting activities. Further, since it is safe with natural product ingredients, it may be usefully used to prevent and improve diabetic diseases and inhibit diabetic complications.

### CROSS-REFERENCE TO RELATED APPLICATIONS

This application claims priority under 35 U.S.C. §119 to Korean Patent Application No. 10-2009-0071210, filed on Aug. 3, 2009, in the Korean Intellectual Property Office, the disclosure of which is incorporated herein by reference in its entirety.

### TECHNICAL FIELD

The following disclosure relates to a composition for treating and preventing diabetes or diabetic complications including an extract of herbal combination as an effective ingredient, and in particular, to a composition for treating and preventing diabetes or diabetic complications including an extract of herbal combination of bitter melon, caterpillar fungus, wolfberry tree bark, white mulberry bark, winged euonymus, kudzu root, Solomon's seal, white atractylodes rhizome, mondo grass rhizome, Japanese cornel fruit and ginseng as an effective ingredient, which is effective in lowering blood glucose level and inhibiting diabetic complications.

### BACKGROUND

Diabetes is considered the most challenging health problem in the 21st century, with more than 170 million patients worldwide. Each year, 3.2 million people are dying of diabetes. In Korea, diabetic patients have increased more than 12-fold in the last 20 years, and it is expected that 1 in 4 Koreans will suffer from diabetes in 2025. Therefore, development of a national policy is urgently needed. A study showing that more than 30% of men and 20% of women in their fifties are at prediabetes stages reveal the severity of diabetes.

Diabetes is a disease affected by acquired factors such as obesity, eating habits, lack of exercise, stress, etc. as well as genetic factors. It is defined as a metabolic disorder induced by defect of insulin secretion from pancreatic cells or failure to use insulin properly. It is accompanied by excessive production of glucose, degradation of body fats and waste of proteins, and results in metabolic disturbance by abnormally accelerating the secretion of glucagon (Abrams, J. J., Ginsberg, H, et al., Metabolism of cholesterol and plasma trigyceride in nonketotic diabetes mellitus. Diabetes, 31, pp. 903-910, 1982).

Thus, diabetes is a chronic metabolic disease leading to disorder of metabolic regulation, hyperglycemia and excretion of glucose through urine. If left alone without adequate treatment or management, it may lead to many complications (Mandrup-Poulsen, T., British Medical Journal, 316, pp. 1221-1225, 1998; Wilson, P. W. F. et al., Am. J. Med., 80, pp. 3-9, 1986).

Diabetes may be classified into insulin-dependent diabetes mellitus (IDDM) and non-insulin-dependent diabetes mellitus (NIDDM) depending on the causes, symptoms and treatment options. In Korea, more than 95% of the diabetic patients suffer from NIDDM. Although diabetes itself may not be a threatening disease, complications resulting from prolonged diabetes, e.g. diabetic complication, retinopathy, cataract, nephropathy, etc., make the patients unable to lead normal lives and may lead to fatal results. This is why diabetes is an important social issue.

The most important goal of diabetic treatment is to manage the blood glucose level as close to the normal level as possible. Management of blood glucose level after meals as well as fasting blood glucose level is important in improving diabetic symptoms and preventing and treating complications. Treatment options include medication, dietary management and exercise (Ref. Jenkins, D. J. A., Woever et. al., Starchy food and glycemic index, Diabetes Care, 11, pp. 149-159, 1988).

However, since the currently available drugs include such chemical substances as insulin and oral hypoglycemic agents, side effects and drug resistances are incessant problems. In this regard, development of an anti-diabetic drug using natural products is of great importance.

Bitter melon (Momordica charantia L.) is a vine of the family Cucurbitaceae. Its original home is not known exactly, but is presumed to originate from Asian tropical regions, including India.

Among the ingredients of the bitter melon, charantin and plant insulin are noteworthy. Charantin is a fat-soluble component promoting the function of the insulin-secreting pancreas. Plant insulin is a kind of peptide serving a similar role as insulin, and is rich in the fruit and seed of the bitter melon (Ref. D. Sathishsekar and S. Subramanian, Biol. Pharm. Bull. 28(6) pp. 978-983, 2005).

Caterpillar fungus (Cordyceps sinensis Sacc) is a compound of the fungus in the dead body of the larva of the ghost moth genus Thitarodes. The caterpillar fungus contains water (10.84%), fats (8.4%), proteins (25.32%), tissue oils (18.53%), carbohydrates (28.90%) and ash (4.10%). Further, it includes quinic acid (˜7%), an isomer of cordycepic acid. It is known to have tonic, immune-enhancing and blood cholesterol-reducing effects and to reduce blood glucose level in nicotinamide- and streptozotocin-induced diabetes (Ref. Lo H. C., et al. Life Sci., 74, pp. 2897-2908, 2004).

It is known that an extract including either or both of bitter melon and caterpillar fungus as an effective ingredient is effective in preventing or improving diabetes.

However, nothing is known or taught about an extract of herbal combination of bitter melon, caterpillar fungus, wolfberry tree bark, white mulberry bark, winged euonymus, kudzu root, Solomon's seal, white atractylodes rhizome, mondo grass rhizome, Japanese cornel fruit and ginseng with regard to prevention or treatment of diabetes.

Through monitoring of body weight change, dietary efficiency, fasting blood glucose level and glucose load in diabetes-induced mice, the inventors found out that an extract of herbal combination of a bitter melon extract, which is known to have a blood glucose-reducing effect, with caterpillar fungus, wolfberry tree bark, white mulberry bark, winged euonymus, kudzu root, Solomon's seal, white atractylodes rhizome, mondo grass rhizome, Japanese cornel fruit and ginseng has a remarkably better anti-diabetic effect than when the herbs are used alone.

### SUMMARY

The present invention is directed to providing a composition for treating and preventing diabetes or diabetic complications effective in reducing blood glucose level and inhibiting diabetic complications, including an extract of herbal combination of bitter melon, caterpillar fungus, wolfberry tree bark, white mulberry bark, winged euonymus, kudzu root, Solomon's seal, white atractylodes rhizome, mondo grass rhizome, Japanese cornel fruit and ginseng as well as a fermented extract of bush clover as an effective ingredient.

In one general aspect, the present invention provides a composition for treating and preventing diabetes or diabetic complications comprising an extract of herbal combination of an ethanol extract of bitter melon, a caterpillar fungus extract and a hot-water extract comprising wolfberry tree bark, white mulberry bark, winged euonymus, kudzu root, Solomon's seal, white atractylodes rhizome, mondo grass rhizome, Japanese cornel fruit and ginseng as well as a fermented extract of bush clover as an effective ingredient.

Other features and aspects will be apparent from the following detailed description, the drawings, and the claims.

### BRIEF DESCRIPTION OF THE DRAWINGS

FIG. 1 shows a toxicity test result for pancreatic tissue after long-term administration of the extract according to the present invention (A: high-dose group, B: intermediate-dose group, C: low-dose group, D: control group).

FIG. 2 shows a toxicity test result for liver tissue after long-term administration of the extract according to the present invention (A: high-dose group, B: intermediate-dose group, C: low-dose group, D: control group).

### DETAILED DESCRIPTION OF EMBODIMENTS

The advantages, features and aspects of the present invention will become apparent from the following description of the embodiments with reference to the accompanying drawings, which is set forth hereinafter. The present invention may, however, be embodied in different forms and should not be construed as limited to the embodiments set forth herein. Rather, these embodiments are provided so that this disclosure will be thorough and complete, and will fully convey the scope of the present invention to those skilled in the art. The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Hereinafter, the present invention will be described in detail.

In the present invention, management of blood glucose level refers to lowering the blood glucose level, unless specified otherwise.

The present invention provides a composition for treating and preventing diabetes or diabetic complications comprising an extract of herbal combination of an ethanol extract of bitter melon, a caterpillar fungus extract and a hot-water extract comprising wolfberry tree bark (1): white mulberry bark (1-10): winged euonymus (1-10): kudzu root (1-10): Solomon's seal (1-10): white atractylodes rhizome (1-5): mondo grass rhizome (1-5): Japanese cornel fruit (1-5): ginseng (1-5), based on weight, as well as a fermented extract of bush clover as an effective ingredient.

The bitter melon is obtained from a water-soluble ethanol extract as powder following drying under reduced pressure. For example, fruit or seed of bitter melon is ground into powder and subjected to reflux in 95% ethanol for 48 hours. The resulting extract is dried under reduced pressure at room temperature. Then, the active ingredient is yielded as yellow powder.

The caterpillar fungus may be from Beauveria bassiana. The caterpillar fungus resulting from the mycelium or fruit body of Beauveria bassiana may be extracted by hot-water extraction and then lyophilized after purification for use as the caterpillar fungus extract.

Preferably, the mixing proportion of the ethanol extract of bitter melon and the caterpillar fungus extract is 4:1-4. They play an important role in reducing blood glucose level.

The wolfberry tree bark (Lycii Radicis cortex) is the root bark of wolfberry, and contains betaine, daucosterine and ascorbic acid. Si, K, Na, Ca, Mg, Fe, D, S, Zn, etc. were identified in ash. The wolfberry tree bark has antifebrile, blood pressure-reducing and blood sugar-lowering effects (Ref. Funayama S. et al., Tetrahedron Lett. 21, pp. 1355-1356, 1980).

The white mulberry bark (Mori Cortex Radicis) is the root bark of mulberry or other plants in the family Moraceae. It contains mulberline (C₂₅H₂₆O₆, 0.15%), mulberlocimene (C₂₅H₂₄O₆, 0.2%), cyclomulberline (C₂₅H₂₄O₆, 0.02%), cyclomulberlocimene (C₂₅H₂₂O₆, 0.016%), etc. and has diuretic, blood pressure-reducing and blood sugar-lowering effects (Ref. Hikino H. et al. Plant Med. 159 pp 160 (1985)).

The winged euonymus (Euonymi Lignum Suberalatum) is obtained by cutting and drying the corky ridges or “wings” of the stem of winged euonymus belonging to the family Celastraceae. The corky wing contains tannins and the root bark contains a gummy substance similar to that of gutta-percha. The winged euonymus inhibits invasion and metastasis of cancer cells and is effective in reducing blood glucose level or urine glucose level in alloxan-induced diabetes in rabbits and lowering body weight (Ref. Kitanaka S. et al. Chem. Pharm. Bull. 44, pp. 615-617, 1996).

The kudzu root (Puerariae Radix) is the root of kudzu (the pea family) with the periderm removed. It contains the isoflavone compounds 4,7-dihydroxyisoflavone, daidzein-7-glucoside, etc. as well as coumarin, and has antifebrile, relaxing, pro-circulatory and blood sugar-lowering effects (Ref. Hsu H. H. et al. Planta Med. 68, pp. 999-1003 (2002)).

The Solomon's seal (Polygonati Rhizoma) is obtained by peeling the skin of the root bark of Solomon's seal or Siberian Solomon's seal and steaming it. It contains a small quantity of alkaloids such as convallarin, convallamarin, etc., as well as asparagine, mannit and minerals such as Mn, Cu, Fe, Mg, Zn and Mo. It has tonifying effect and sugar-lowering effect in streptozotocin-induced diabetes in mice (Ref. Kato A. Miura T., Planta Med. 60 pp. 201-203 (1994)).

The white atractylodes rhizome (Atractylodis Rhizoma Alba) is the root bark of Atractylodes japonica or Atractylodes ovata with the periderm removed. The root bark contains essential oil (˜1.5%) as well as carotene, inulin, gum resin, saponin, coumarin, or the like. It has stomach-relaxing, diuretic, relaxing and blood sugar-lowering effects (Ref. Konno C. et al. Planta Med. 51, pp. 102 (1985)).

The mondo grass rhizome (Liriopis Tuber) refers to the tuberous root of mondo grass. The dry tuberous root contains monosaccharides (˜10%) and low-molecular-weight polysaccharides (˜43%). Further, it contains β-sitosterol, ruscogenin, and so forth. It has antifebrile, cough-suppressing and anti-inflammatory effects. The aqueous extract has a continued blood sugar-lowering effect (Ref. Lin Y C et al. Am. J. Chin. Med. 31, pp. 354-579 (2003)).

The Japanese cornel fruit (Corni Fructus) refers to the pulp of Japanese cornel of the family Cornaceae with the seeds removed. It contains gallic acid, malic acid, tartaric acid as well as morroniside and the saponin loganin. It has diuretic, blood pressure-reducing, tonifying and blood sugar-lowering effects (Ref. Jeng H. et al. Am. J. Chin. Med. 25, pp. 301-306 (1997)).

The ginseng (Ginseng Radix Alba) refers to the root of ginseng of the family Araliaceae with the rootlets and the corky layer removed. The root contains saponins, essential oil, amino acids, alkaloids, carbohydrates, resin, trace elements, and so forth. It has tonifying, immune-enhancing, pro-CNS, pro-cardiovascular, blood sugar-lowering, lipid-metabolizing, anti-cancer and pro-adrenocortical actions (Ref. Yokozawa T. et al. Chem. Pharm. Bull. 33, pp. 869-872 (1985)).

The composition of the present invention comprises an extract of herbal combination of an ethanol extract of bitter melon, a caterpillar fungus extract and a hot-water extract of wolfberry tree bark, white mulberry bark, winged euonymus, kudzu root, Solomon's seal, white atractylodes rhizome, mondo grass rhizome, Japanese cornel fruit and ginseng, the hot-water extract comprising wolfberry tree bark (1): white mulberry bark (1-10): winged euonymus (1-10): kudzu root (1-10): Solomon's seal (1-10): white atractylodes rhizome (1-5): mondo grass rhizome (1-5): Japanese cornel fruit (1-5): ginseng (1-5), based on weight.

For example, the hot-water extract may be prepared by finely cutting wolfberry tree bark (250 g), white mulberry bark (280 g), winged euonymus (200 g), kudzu root (250 g), Solomon's seal (230 g), white atractylodes rhizome (100 g), mondo grass rhizome (100 g), Japanese cornel fruit (100 g) and ginseng (100 g), immersing in water (20 L) at room temperature for 2 hours, heating at 80° C. for 3 hours, further heating for 1.5 hours in water (15 L), further heating for 1 hour in water (15 L), and then concentrating the resulting extract at 80° C. under reduced pressure.

The extract according to the present invention of herbal combination comprises the ethanol extract of bitter melon, the caterpillar fungus extract and the hot-water extract, with a mixing proportion of 4:1-4:2-5, based on weight.

The fermented extract of bush clover may be prepared by inoculating a bush clover extract (20-40 wt %) with Aspergillus oryzae and then performing solid-state fermentation. The bush clover extract may be prepared by extracting bush clover with water, ethanol or a mixture solvent thereof and then sterilizing at high temperature above the boiling point of ethanol (78.3° C.).

The whole bush clover including root is cleanly washed and then cut finely. One or more sweet and tasty fruit(s) selected from a group consisting of pear, apple, persimmon and wolfberry fruit may be included. These fruits improve preference of the extract of bush clover, which tastes bitter and hot.

The finely cut bush clover as well as the fruit(s) are ground into powder and then aged after adding ethanol. It is to be noted that the bush clover, which is used as a functional well-being herb, has decreased efficiency unless it is aged with ethanol. Thus, differently from the process of preparing ordinary tea or drinks, the process of naturally aging the bush clover in ethanol for 5 to 10 days is necessary.

Although ethanol is evaporated during the aging of the bush clover extract, the extract is not suitable as a drink because of the alcoholic and bitter taste. To solve this problem, the inventors of the present invention heat the bush clover extract at high temperature above the boiling point of ethanol (78.3° C.) for about 5 hours to remove the alcohol groups and sterilize the extract.

For example, bush clover is cut finely and, after adding 95% ethanol, aged for 10 days. The aged bush clover extract is sieved through a sieve or cloth to remove solid impurities. The recovered bush clover extract is heated at high temperature above the boiling point of ethanol (78.3° C.) for 5 hours to remove the alcohol groups and sterilize the extract. About 20 g of thus obtained bush clover extract is inoculated with 0.1% aspergillus (Aspergillus oryzae, KFRI 00888 (ATCC 22887)). Then, the composition according to the present invention is prepared by culturing in solid state under the condition of 30-32° C., and 70-85% humidity for 36 to 42 hours. 0.001% biotin may be used to promote the cultivation.

It is considered that, during the fermentation of the bush clover extract, the effective ingredients are converted into various useful components. In particular, various enzyme components are produced through the fermentation, proteins are degraded to low-molecular-weight products, vitamins are synthesized, and functional components are converted into activated forms. Diabetes, which results from glucose metabolic dysfunction, may be improved or prevented by the activated components in the solid-state fermentation product, which promote metabolism and activate enzymatic and cellular functions.

In the present invention, the diabetes includes type 1 diabetes and type 2 diabetes, and the diabetic complication may be neuropathy, diabetic retinopathy, nephropathy, sexual dysfunction, skin disease, hypertension, arteriosclerosis, stroke, heart disease or gangrene. That is to say, the composition according to the present invention for treating and preventing diabetes or diabetic complications increases secretion of insulin from the pancreas of a diabetic mammal, increases insulin level in the pancreas, increases fasting blood glucose level, and improves glucose tolerance.

The present invention also provides a pharmaceutical composition comprising an extract of herbal combination comprising an ethanol extract of bitter melon, a caterpillar fungus extract and a hot-water extract comprising wolfberry tree bark, white mulberry bark, winged euonymus, kudzu root, Solomon's seal, white atractylodes rhizome, mondo grass rhizome, Japanese cornel fruit and ginseng as an effective ingredient.

The pharmaceutical composition may further comprise an adequate carrier, excipient or diluent commonly used for the preparation of pharmaceutical compositions. The carrier, excipient or diluent may include sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate and mineral oil.

The pharmaceutical composition comprising the extract of the present invention may be prepared into oral administration formulations such as powder, granule, tablet, capsule, suspension, emulsion, syrup, aerosol, etc. formulation for external application, suppository or sterile injection solution according to common methods.

When preparing the formulations, a commonly used diluent or excipient such as filler, extender, binder, humectant, disintegrant, surfactant, etc. may be included. Solid formulations for oral administration include tablet, pill, powder, granule, capsule, etc. The solid formulation may be prepared by mixing the extract or its powder with at least one excipient(s), e.g. starch, calcium carbonate, gelatin, etc. Further, in addition to simple excipient, lubricant such as magnesium stearate or talc may be used. Liquid formulations for oral administration include suspension, liquid for internal use, emulsion, syrup, etc. In addition to commonly used simple diluent such as water and liquid paraffin, various excipients, e.g. humectant, sweetener, aromatic, preservative, etc., may be included. Formulations for parenteral administration include sterile aqueous solution, non-aqueous solution, suspension, emulsion, lyophilized preparation and suppository. The non-aqueous solution or suspension may be propylene glycol, polyethylene glycol, vegetable oil such as olive oil, injectable ester such as ester, or the like. A base for the suppository may include witepsol, macrogol, Tween 61, cacao oil, laurine oil, glycerogelatin, or the like.

A preferred administration dose of the extract of the present invention varies depending on the patient's physical conditions and body weight, severity of disease, formulation type, administration route and administration period, and may be determined adequately by those skilled in the art. To attain a desired effect, the extract of the present invention may be administered at a dose of 0.01 to 20 g/kg, preferably 0.01 to 10 g/kg, once or several times a day. In the pharmaceutical composition, the extract of the present invention may be included in an amount of 0.1 to 50 wt % based on the total weight of the composition.

The pharmaceutical composition of the present invention may be administered to mammals including mouse, cattle and human via various routes. All administration routes may be expected including, for example, oral, rectal, intravenous, intramuscular, subcutaneous, intrauterine and intracerebroventricular routes.

The present invention further provides a health food for lowering blood glucose level comprising an extract of herbal combination of an ethanol extract of bitter melon, a caterpillar fungus extract and a hot-water extract comprising wolfberry tree bark, white mulberry bark, winged euonymus, kudzu root, Solomon's seal, white atractylodes rhizome, mondo grass rhizome, Japanese cornel fruit and ginseng as an effective ingredient.

The extract may be used alone or in combination with other food ingredients, according to common, suitable methods. The mixing proportion of the effective ingredients may be determined adequately depending on purposes (prevention, health improvement or treatment). In general, when preparing foods or drinks, the extract of herbal combination of the ethanol extract of bitter melon, the caterpillar fungus extract and the hot-water extract comprising wolfberry tree bark, white mulberry bark, winged euonymus, kudzu root, Solomon's seal, white atractylodes rhizome, mondo grass rhizome, Japanese cornel fruit and ginseng is added in an amount of 10 to 90 wt %, preferably 30 to 70 wt % based on the total weight.

However, in case of long-term intake for health improvement or health management, the quantity may be smaller. Also, since the effective ingredients have no safety problems, they may be used in larger quantities.

The kind of the food is not particularly limited. For example, any common health food including meat, sausage, chocolate, candy, snack, confectionery, pizza, noodle, gum, dairy products including ice cream, soup, beverage, drink, tea, alcoholic beverage, vitamin complex, or the like are included.

Since the composition according to the present invention for treating and preventing diabetes or diabetic complications increases secretion of insulin in type 2 diabetic model animals, improves insulin resistance, exhibits strong blood sugar-lowering activity and diabetic complication inhibition activity, and is safe as natural product ingredients, it may be usefully used to prevent and improve diabetic diseases and inhibit diabetic complications.

### EXAMPLES

The examples and experiments will now be described. The following examples and experiments are for illustrative purposes only and not intended to limit the scope of this disclosure.

### Preparation Example

### (1) Preparation of Bitter Melon Extract

After removing impurities from fruit (1 kg) and seed (1 kg) of bitter melon and washing with water, the fruit and seed were pulverized into 100-400 mesh powder. After refluxing in 95% ethanol (20 L) for 48 hours, the resulting extract was dried under reduced pressure at room temperature. The active ingredient (30 g) was yielded as yellow powder.

### (2) Preparation of Caterpillar Fungus Extract

After removing impurities from Beauveria bassiana caterpillar fungus (1 kg) and washing with water, the caterpillar fungus was cut finely, immersed in water (20 L) at room temperature for 2 hours, heated at 80° C. for 3 hours, heated in water (15 L) for 1.5 hours, and then heated in water (15 L) for 1 hour.

The resulting extract was concentrated under reduced pressure at 80° C., purified and then lyophilized. The caterpillar fungus extract (10 g) was yielded.

### (3) Preparation of Hot-Water Extract

After removing impurities from wolfberry tree bark (250 g), white mulberry bark (280 g), winged euonymus (200 g), kudzu root (250 g), Solomon's seal (230 g), white atractylodes rhizome (100 g), mondo grass rhizome (100 g), Japanese cornel fruit (100 g) and ginseng (100 g) washing with water, the mixture (1610 g) was immersed in water (20 L) at room temperature for 2 hours, heated at 80° C. for 3 hours, heated in water (15 L) for 1.5 hours, and then heated in water (15 L) for 1 hour.

The resulting extract was concentrated under reduced pressure at 80° C. The hot-water extract (250 g) was yielded.

### (4) Preparation of Fermented Bush Clover

Bush clover (1 kg) was washed cleanly and cut finely. After adding 95% ethanol (20 L), the bush clover was aged for 10 days. The aged bush clover extract was sieved through a 20-50 mesh sieve or cloth to separate solidified impurities. The recovered bush clover extract was sterilized at high temperature above the boiling point of ethanol (78.3° C.) for 5 hours to remove the alcohol groups.

Thus obtained bush clover extract (20 g) was inoculated with Aspergillus oryzae (KFRI 00888 (ATCC 22887), 0.1%) and subjected to solid-state fermentation in an incubator of 30-32° C. and humidity 70-85% for 36 to 42 hours. Biotin (0.001%) was added to promote the fermentation.

### Example

### Preparation of Composition for Treating and Preventing Diabetes or Diabetic Complications

The ethanol extract of bitter melon (30 g), the caterpillar fungus extract (10 g), the hot-water extract (250 g) and the fermented extract (10 g) were homogeneously mixed at 40° C. or below. The composition for treating and preventing diabetes or diabetic complications (300 g) was prepared.

### Test Example 1

### Acute Toxicity Test

1. Test System

(1) Species: Specific pathogen free (SPF) mouse

(2) Source of supply: Department of Test Animal Breeding, Jilin Veterinary University Research Center

(3) Reason of test system selection

Mouse is widely used for acute toxicity test. Since abundant data are available for SPF mouse, analysis and evaluation of test result can be made using them.

(4) Age and body weight

| | | |
|---|---|---|
| Male | Age upon acquirement | 4 weeks |
| | Number of animals upon acquirement | 12 |
| | Body weight upon acquirement | 79.2 to 97.5 g |
| | Age at initiation of administration | 5 weeks |
| | Number of animals at initiation of | 10 |
| | administration | |
| | Body weight at initiation of administration | 119.3 to 136.1 g |
| Female | Age upon acquirement | 4 weeks |
| | Number of animals upon acquirement | 12 |
| | Body weight upon acquirement | 79.4 to 96.4 g |
| | Age at initiation of administration | 5 weeks |
| | Number of animals at initiation of | 10 |
| | administration | |
| | Body weight at initiation of administration | 99.8 to 118.7 g |

(5) Quarantine and accommodation

Appearance of the animals was inspected upon acquirement. After accommodation for 7 days in a test laboratory, only the health animals were subjected to the test.

2. Breeding Environment

(1) Environmental condition

The test was carried out in Intermediate-Sized Animal Lab 2 of the Toxicity Research Center, Korea Research institute of Chemical Technology, under the condition of 23±30° C., relative humidity 55±15%, lighting period 12 hours (8 a.m. to 8 p.m.) and illumination 150-300 lux. All the operators performed the test wearing steam-sterilized (121° C., 20 min) working clothes, hood, mask and gloves.

(2) Monitoring of breeding environment

During the test period, temperature and humidity of the animal labs were automatically measured every hour. Illumination and other environmental conditions were monitored regularly (once in 3 months). No change that might affect the test was observed.

(3) Breeding cage, breeding density and identification of breeding cage

During the test period, the animals were bred in a stainless-steel breeding cage (220w×410L×200H mm). Four animals were kept in one cage during the accommodation, and five during the administration. The breeding cages were identified by attaching ID cards (control group: white, T1: yellow).

(4) Feed and water

a) Feed

Solid feed for test animals (Jilin Feed Industry) was given freely after radiation sterilization (2.0 Mrad). No significant factor that might affect the test was observed upon microbiological and contamination tests by the present inventors and the feed supplier.

b) Supply of Drinking Water and Test of Contamination

Tap water was given freely as drinking water after UV sterilization. Water quality examination was performed before providing the water. No significant factor that might affect the test was observed.

3. Administration Dose and Test Groups

(1) Administration dose

When preliminary test was carried out with the plant extract, no case of death was observed at the maximum dose of 2000 mg/kg. Thus, 2000 mg/kg was set as the highest dose of limit dose test. Only the vehicle was provided for the control group. Since there was no limit dose standard available in “Rules on Toxicity Test” of China's Health and Welfare Ministry, “The Guidelines on Non-clinical Test of Medical Products (1997)” of Japan's Ministry of Health and Welfare was referred to.

(2) Test groups, administration concentration and dose

| | | | | Admnistration | |
|---|---|---|---|---|---|
| | | Number of | Animal | quantity | Administration |
| Group | Sex | animals | No. | (mL/kg) | dose (mg/kg) |
| Control | Female | 5 | 1-5 | 20 | 0 |
| | Male | 5 | 11-15 | 20 | 0 |
| T1 | Female | 5 | 6-10 | 20 | 2000 |
| | Male | 5 | 16-20 | 20 | 2000 |

(3) Grouping and identification of animals

Animals were grouped as follows. First, the animals that were decided to be healthy during the accommodation period were weighed. Then, each 10 males and females close to the average weight were selected. The selected animals were randomly grouped into two groups for each sex. The animals were identified by means of pigmentation marking and ID cards.

4. Administration of Test Substance

(1) Preparation of administration solution

The administration solution was prepared by dissolving the test substance in physiological saline for injection at 2000 mg/20 mL/kg. For the control group (vehicle control), 0.5% CMC was administered.

(2) Administration route and method

After fasting overnight, the administration solution was forcibly administered orally using a syringe equipped with a sonde for oral administration.

(3) Reason for selection of administration route

Oral administration was selected because clinical trial is planned for oral administration.

(4) Administration time and period

The test substance was administered once in the morning of the administration day.

(5) Calculation of administration dose

Administration dose (20 mL/kg) was calculated based on the body weight measured on the administration day.

5. Observations and Examinations

(1) General symptoms and animal death

General symptoms and animal death were observed every hour from hours 1 to 6 on the administration day and more than once from the next day until day 14.

(2) Body weight

Body weight was measured before administration and days 1, 3, 7 and 14 after the onset of administration.

(3) Autopsy

After anesthetizing with CO₂ gas and cutting the abdomen open, all the internal organs were examined with naked eyes.

6. Statistical Analysis

Statistical analysis was performed according to the SOP of the Jilin Veterinary University Toxicity Research Center.

7. Result

No significant symptom was observed in any test group. All the test groups showed normal body weight increase on days 1, 3, 7 and 14 after the onset of administration.

No death was observed in either the control group or the 2000 mg/kg administration group during the test period. LD₅₀ (the dose required to kill 50% of the animals) of the test substance was over 2000 mg/kg for both male and female animals. No abnormality was observed after autopsy.

To conclude, as a result of performing the single oral-administration toxicity test of the composition according to the present invention for treating and preventing diabetes or diabetic complications on male and female SD mice, no significant change in mortality, general symptoms, body weight or autopsy findings resulting from the test substance was observed.

### Test Example 2

### Long-Term Toxicity Test

1. Test Substance

The test substance was prepared by dissolving the powder (1 g, corresponding to 10.6 g of herb) of the composition for treating and preventing diabetes or diabetic complications according to the present invention in distilled water.

2. Test Animal

Male and female SD mice weighing 95 to 102 g, acquired from Xian Medical University Animal Experiment Center (Approval No. 07-005), were used.

160 SD mice were randomly grouped into 4 groups, with 20 males and 20 females per each group. For a high-dose group, a 15.0% suspension of the test substance (3.0 g, 50 times of clinical dose) was orally administered at 20 mL/kg. For an intermediate-dose group, a 7.5% suspension of the test substance (1.5 g) was orally administered at 20 mL/kg. For a low-dose group, a 1.5% suspension of the test substance (0.3 g) was orally administered at 20 mL/kg. For the control group, distilled water was administered.

The animals were bred in an air-conditioned facility at 18-24° C. and relative humidity 45-70%. Ventilation was provided and lighting period was 12 hours a day. Standard solid feed produced from Xian Medical University Animal Experiment Center was given and tap water was given as drinking water. The animals of different sexes were kept in different cages.

Five mice were kept in one breeding cage. The breeding cage was cleaned once in 3 days. The following experiment was carried out after a week of accommodation under the above conditions.

SD mice were selected for the toxicity test because abundant data are available for them.

3. Test Method (Administration)

The test substance was orally administered once a day. Examination was made after administration for 3 months.

4. Examination

(1) General status and body weight

For all the tested animals, neurological status, behavioral status, secretion, eating habit, feces, hair, skin, etc. were observed. Administration dose was controlled once in 2 weeks according to the result of body weight measurement.

(2) Hematological examination

For all the tested animals, blood was taken from the femoral artery 3 months after the administration. Blood proteins (Hb), red blood cells (RBC), white blood cells (WBC) and platelets (Pt) were examined.

(3) Hematobiochemical examination

For all the tested animals, blood was taken from the femoral artery 3 months after the administration. Concentrations of various substances (ALT, AST, ALP, TP, ALB, BUN, CRE, T-BIL, GLU) in the blood were examined

(4) Urine examination

Urine was examined for all the tested animals. Color, urine glucose, urine protein, red blood cells, white blood cells, etc. were examined

(5) Electrocardiogram (ECG)

Standard ECG was monitored for all the tested animals during the test period.

(6) Dissection (major organs)

For all the tested animals, heart, liver, spleen, lungs, kidneys, thymus, suprarenal glands, testicles and ovary were examined.

(7) Histopathological examination

For all the tested animals, heart, liver, spleen, lung, kidney, stomach, duodenal, jejunal, colic, bladder, mesenteric lymph node, testicular, epididymal, ovarian, uterine, sterna (medullar), cerebral, pituitary, suprarenal gland, thymus and thyroid tissues were examined.

5. Result

After 13 weeks of administration, general status was good. Hair status, neurological status, behavioral status and secretion from eyes and nose were also normal. Eating habit observed for 24 hours was also normal. Body weight increased gradually but there was no significant difference from the control group.

In order to study hematological and hematobiochemical effects, hematological and hematobiochemical examinations were carried out after anesthetizing the mice by subcutaneous injection of pentobarbital and taking blood from the femoral artery. As seen from Table 1, there was no significant difference between the administration groups. Also, there was no remarkable difference from the control group.

TABLE 1
Result of hematological examination after 3 months of administration

| | Number of | | RBC | WBC | WBC type | | Platelet |
|---|---|---|---|---|---|---|---|
| Groups | animals (n) | Blood protein | (×10¹²/L) | (×10⁹/L) | granulocyte | lymphocyte | (×10⁹/L) |
| Control | 20 | 141.5 ± 12.3 | 6.6 ± 0.7 | 8.5 ± 2.2 | 0.12 ± 0.047 | 0.86 ± 0.087 | 225.2 ± 74.5 |
| High-dose | 20 | 142.9 ± 17.4 | 6.7 ± 0.8 | 9.8 ± 3.0 | 0.15 ± 0.040 | 0.82 ± 0.056 | 238.7 ± 54.1 |
| Intermediate- | 20 | 146.4 ± 14.9 | 6.9 ± 1.0 | 9.7 ± 2.8 | 0.16 ± 0.062 | 0.87 ± 0.079 | 211.8 ± 51.9 |
| dose | | | | | | | |
| Low-dose | 20 | 140.5 ± 10.2 | 6.8 ± 1.0 | 8.8 ± 1.9 | 0.14 ± 0.051 | 0.88 ± 0.068 | 199.1 ± 61.4 |

Hematobiochemical effect was evaluated by examining aminotransferase, liver function, kidney function and blood glucose level. As seen from Table 2, the high-dose administration group showed remarkably decreased blood glucose level, with significant difference from the control group. Other biochemical factors were normal and there was no statistically significant difference from the control group.

TABLE 2
Hematobiochemical effect after 3 months of administration

| | Number of | | | | | | | | | |
|---|---|---|---|---|---|---|---|---|---|---|
| | animals | ALT | AST | ALP | TP | ALB | GLU | CRE | BUN | T-BIL |
| Groups | (n) | (U/L) | (U/L) | (U/L) | (g/L) | (g/L) | (mmol/L) | (μmol/L) | (mmol/L) | (mmol/L) |
| Control | 20 | 13.2 ± 6.4 | 75.5 ± 19.4 | 119.7 ± 29.4 | 89.1 ± 10.4 | 46.7 ± 8.7 | 6.00 ± 1.51 | 79.9 ± 28.6 | 13.2 ± 3.0 | 5.2 ± 3.4 |
| High-dose | 20 | 12.6 ± 5.1 | 69.8 ± 20.8 | 128.6 ± 33.1 | 85.2 ± 9.2 | 47.4 ± 8.1 | 5.49 ± 0.98 | 80.8 ± 30.9 | 12.8 ± 2.9 | 5.4 ± 3.7 |
| Intermediate- | 20 | 10.9 ± 6.2 | 76.9 ± 20.0 | 140.4 ± 40.2 | 88.1 ± 11.5 | 42.8 ± 7.7 | 5.84 ± 1.09 | 85.4 ± 39.4 | 13.1 ± 2.8 | 5.9 ± 2.9 |
| dose | | | | | | | | | | |
| Low-dose | 20 | 11.8 ± 5.2 | 73.9 ± 22.8 | 126.8 ± 30.9 | 92.6 ± 8.8 | 50.0 ± 10.1 | 5.79 ± 1.41 | 90.2 ± 36.1 | 12.9 ± 2.5 | 6.1 ± 3.0 |

As seen from Table 3, all the tested animals showed normal urine status. Urine glucose, urine protein, RBC or WBC was not observed. This confirms that the test substance has no effect on the mouse urine.

TABLE 3
Result of urine examination

| | Number of | | | | | |
|---|---|---|---|---|---|---|
| | animals | | Urine | Urine | | |
| Groups | (n) | Color | glucose | protein | RBC | WBC |
| Control | 10 | Lemon | — | — | — | — |
| High-dose | 10 | Lemon | — | — | — | — |
| Intermediate-dose | 10 | Lemon | — | — | — | — |
| Low-dose | 10 | Lemon | — | — | — | — |

As a result of ECG monitoring after 3 months of administration of the test substance, there was no difference from the control group in pulse rate and pulse waves including S- and T-waves. When heart, liver, spleen, brain, lungs, kidneys, stomach, jejunum, colon, bladder, gallbladder, suprarenal glands, thymus, testicles, uterus, brain, pituitary, abdominal cavity and thoracic cavity of the test animals were examined with naked eyes, neither abnormality in shape or color nor lesion was observed.

Heart, liver, spleen, kidneys, suprarenal glands, thymus, testicles and ovary were weighed (g/100 g body weight or mg/100 g body weight)

As seen from Table 4, all the organs were within the normal range.

TABLE 4
Weight of organs

| | Number of | | | | | | Suprarenal | | | |
|---|---|---|---|---|---|---|---|---|---|---|
| | animals | | | | | | gland | Thymus | Testes | Ovary |
| Groups | (n) | Heart | Liver | Spleen | Lungs | Kidneys | (mg %) | (mg %) | (n = 10) | (n = 10) |
| Control | 20 | 0.30 ± 0.03 | 3.3 ± 0.20 | 0.25 ± 0.04 | 0.80 ± 0.14 | 0.29 ± 0.07 | 14.9 ± 3.8 | 110.5 ± 29.3 | 0.50 ± 0.11 | 0.35 ± 0.07 |
| High-dose | 20 | 0.30 ± 0.02 | 3.3 ± 0.31 | 0.24 ± 0.05 | 0.79 ± 0.18 | 0.28 ± 0.08 | 15.0 ± 4.0 | 113.1 ± 30.4 | 0.51 ± 0.12 | 0.34 ± 0.06 |
| Intermediate- | 20 | 0.31 ± 0.05 | 3.2 ± 0.29 | 0.25 ± 0.03 | 0.80 ± 0.17 | 0.29 ± 0.09 | 14.8 ± 4.2 | 109.8 ± 31.0 | 0.55 ± 0.09 | 0.37 ± 0.05 |
| dose | | | | | | | | | | |
| Low-dose | 20 | 0.30 ± 0.06 | 3.1 ± 0.33 | 0.25 ± 0.03 | 0.78 ± 0.20 | 0.27 ± 0.06 | 14.9 ± 4.7 | 107.4 ± 31.1 | 0.52 ± 0.10 | 0.36 ± 0.05 |

The mouse organs were subjected to histopathological examination. 6 mice were selected from each group.

Heart, liver, spleen, lung, kidney, stomach, duodenal, jejunal, colic, bladder, mesenteric lymph node, testicular, epididymal, ovarian, uterine, sterna (medullar), cerebral, pituitary, suprarenal gland, thymus and thyroid tissues were examined.

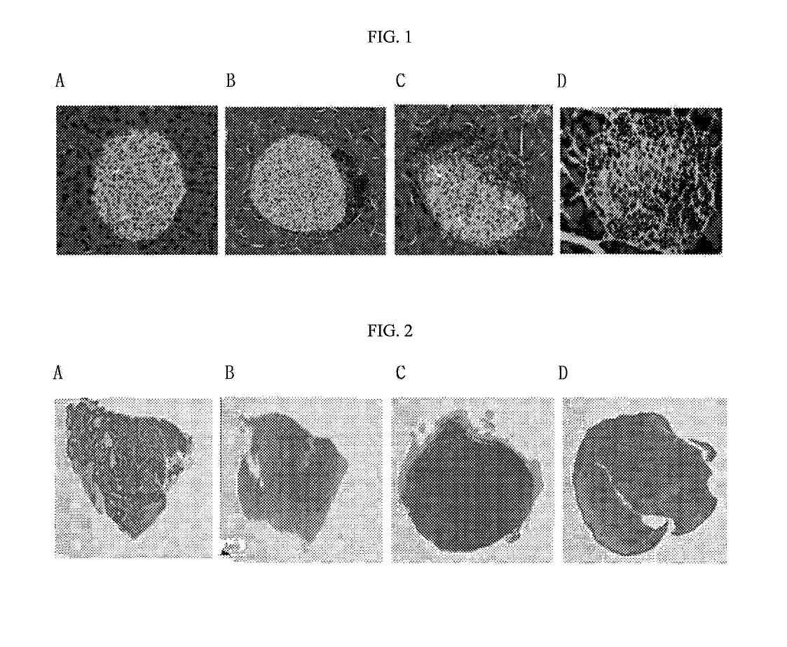

The tissues were fixed in 10% formaldehyde solution and prepared into paraffin sections. After HE staining, they were observed under a microscope. As seen from FIG. 1 and FIG. 2, the tissues of the test groups showed clear structures, with no pathological changes including congestion, edema, degeneration, necrosis, cellular infiltration, or the like.

Thus, the test substance was confirmed to lead to no pathological results. After 13 weeks of administration, no special toxicity was observed. A safe administration dose was calculated as 3.0 g/kg body weight/day.

### Test Example 3

### Blood Sugar-Lowering Effect

The following experiment was carried out in order to evaluate the blood sugar-lowering effect of the composition for treating and preventing diabetes or diabetic complications (test substance).

Streptozotocin (STZ, Sigma. Co., USA) dissolved in 0.85% saline was repeatedly administered to mice acquired from the Korea Laboratory Animal Center for 5 days in order to artificially induce diabetes. After administration of the test substance, change in blood glucose level was observed to evaluate the therapeutic effect of the composition of the present invention.

1. Test Method

The animals acquired from the Korea Laboratory Animal Center were kept in clean rooms maintained at 22±2° C., humidity 55±5% and 12L/12D lighting.

The animals were grouped into 4 groups: a first control group without administration of streptozotocin, a second control group with streptozotocin (60 mg/kg) abdominally administered 3 to 5 times, a third control group with streptozotocin and placebo (0.85% saline) administered, and a test group with the substance of the third control group plus the test substance (267 mg/mL/kg) administered.

The test substance was administered for 23 days, and blood glucose level, urine glucose and body weight were measured every 4 days.

Blood glucose level was measured with the blood taken from the tail vein using a blood glucose meter (Accutrend, Green Cross). Measurement was made with 500 mg/dL as maximum.

2. Result

As seen from Table 5, 9 out of the 10 tested mice were diagnosed with diabetes (blood glucose level ≧300 mg/dl) in the second control group. The third control group had 8 diabetic mice. In contrast, the onset of diabetes was prevented in the test group.

TABLE 5
Blood glucose level in mice administered with streptozotocin

| | |
|---|---|
| Test No. | Mean blood |

| Groups | Day | 1 | 2 | glucose level |
|---|---|---|---|---|
| Control 1 | 0 | 203 | 200 | 201.5 |
| | 4 | 187 | 150 | 168.5 |
| | 8 | 215 | 190 | 202.5 |
| Control 2 | 0 | 205 | 191 | 198.0 |
| | 4 | 326 | 308 | 317.0 |
| | 8 | 406 | 500 | 453.0 |
| Control 3 | 0 | 203 | 175 | 189.0 |
| | 4 | 318 | 314 | 316.0 |
| | 8 | 470 | 500 | 485.0 |
| Test | 0 | 221 | 265 | 243.0 |
| | 4 | 194 | 169 | 181.5 |
| | 8 | 202 | 189 | 195.5 |

### Test Example 4

### Effect on Non-Insulin-Dependent Diabetes Mellitus (NIDD)

In order to evaluate the effect of the test substance on type 2 (non-insulin-dependent) diabetes, the test substance was administered to 51 NIDD diabetic patients living in Xian of Shanxxi Province and Changchun of Jilin Province in China, during the period from December 2007 until September 2008. Then, clinical therapeutic effect was evaluated.

1. Selection of Subjects

1) Subject selection standard (WHO Standard of 1998): After blood glucose level measurement, those who satisfy any one of (1) to (3) are diagnosed as diabetic patients.

(1) Fasting blood glucose level ≧7.8 mmol/L (140 mg/100 mL), or post-prandial glucose level ≧11.1 mmol/L (200 mg/100 mL).

(2) Fasting blood glucose level <7.8 mmol/L, but blood glucose level ≧11.1 mmol/L (200 mg/100 mL) upon OGTT with glucose 75 g.

(3) Blood glucose level 1 hour after OGTT ≧11.1 mmol/L, blood glucose level 2 hours after OGTT ≧11.1 mmol/L, or fasting blood glucose level ≧7.8 mmol/L.

2) Standard of Test Subject Selection

Those who meet one of the followings are excluded from the test.

(1) Although the blood glucose level is higher than normal level as compared with before administration of the test substance, fasting blood glucose level is higher than 7.8 mmol/L (140 mg/100 mL) or 2 hour blood glucose level is lower than 11.1 mmol/L (200 mg/100 mL) as a result of diet or exercise.

(2) Those who fail to follow the diet regimen or drug prescription regimen.

(3) Those with severe complications or other medical history.

(4) Those whose diabetes has begun within 1 month or those with acidism or infections.

(5) Those who dropped out or those with insufficient data.

(6) Patients with insulin-dependent diabetes (IDD)

(7) Those who are 15 years old or younger, pregnant or in childbed.

3) Generals

(1) Age and sex

| | Age | | | | | | | |
|---|---|---|---|---|---|---|---|---|
| Sex | <30 | 31-40 | 41-50 | 51-60 | 61-70 | 71-80 | >80 | Total |
| Male | 0 | 0 | 1 | 12 | 15 | 3 | 0 | 31 |
| Female | 0 | 1 | 1 | 4 | 8 | 5 | 1 | 20 |
| Total | 0 | 1 | 2 | 16 | 23 | 8 | 1 | 51 |

(2) History of diabetes

| | History | | | | | |
|---|---|---|---|---|---|---|
| | | | | | 4-5 | >5 |
| | <1 year | 1-2 years | 2-3 years | 3-4 years | years | years |
| Number of | 1 | 2 | 5 | 8 | 16 | 19 |
| cases | | | | | | |

2. Test Method

1) Generals

(1) Physical checkup

(2) Routine blood sugar and blood urine tests

(3) Heart, kidney, liver and blood lipid tests for some patients

2) Tests

(1) Medical history is checked in detail. Further, diet, exercise, mental sate and knowledge about diabetes are checked.

(2) Fasting and 2 hour blood glucose level tests (every 10 to 15 days after onset of administration)

(3) Glucose tolerance test (OGTT)

(4) Insulin release test (INSRT)

(5) Aminofructose measurement

Administration period is basically 60 days. (1) Blood glucose level test and (2) glucose tolerance test are performed necessarily, and others are performed considering the patient's status. Fasting blood glucose level and urine glucose level, and 2 hour blood glucose level and urine glucose level are measured every 10 to 15 days after onset of administration.

After the onset of administration, the following diet regimen is applied.

Standard of diet regimen

1) General patients: 250 to 300 g/day
2) Office workers: 300 to 350 g/day
3) Manual workers: 400 to 500 g/day
4) In addition to the above main diet, insufficient calorie may be
supplemented with other foods.

3) Treatment and Observation

(1) Administration method: Administration was made orally, 3 times a day, 4 capsules once, between 30 minutes before and 30 minutes meals. Later, the administration dose was adjusted with 3-4 capsules a day depending on the blood sugar-lowering effect.

(2) Treatment period: Administration was made for 60 days. Fasting and 2 hour blood glucose level and urine glucose level were measured every 10 to 15 days.

4) Evaluation Standard

According to the Chinese Diabetes Association standard and China's Health and Welfare Ministry standard,

(1) Significantly effective:
Disappearance of general symptoms
Fasting blood glucose level <7.2 mmol/L (130 mg/100 mL)
2 hour blood glucose level <8.3 mmol/L (150 mg/100 mL)
Urine glucose level within 24 hours <10 (g)
Blood glucose level and urine glucose level within 24 hours decreased by more than 30%

(2) Effective:
Improvement of general symptoms
Fasting blood glucose level <8.3 mmol/L (150 mg/100 mL)
2 hours blood glucose level <10 mmol/L (180 mg/100 mL)
Urine glucose level within 24 hours <25 (g)
Blood glucose level and urine glucose level within 24 hours decreased by more than 10%

(3) Ineffective:
No improvement of general symptoms and decrease of blood glucose level and urine glucose level failing to meet the above standard

3. Test Result

Change in blood glucose level before and after administration is shown in Tables 6 and 7.

TABLE 6
Blood glucose level before administration (mmol/L)

| Blood glucose level | Male | Female | Total |
|---|---|---|---|
| 7.2-8.0 | 1 | — | 1 |
| 8.1-9.0 | — | — | — |
| 9.1-10.0 | 2 | 1 | 3 |
| 10.1-11.0 | — | 1 | 1 |
| 11.1-12.0 | 1 | 1 | 1 |
| 12.1-13.0 | 6 | 4 | 10 |
| 13.1-14.0 | 6 | 1 | 7 |
| 14.1-15.0 | — | — | — |
| 15.1-16.0 | 4 | 1 | 5 |
| 16.1-17.0 | — | — | — |
| 17.1-18.0 | — | 1 | 1 |
| 18.1-19.0 | 5 | 2 | 7 |
| 19.1-20.0 | — | — | — |
| 20.1-21.0 | — | — | — |
| 21.1-22.0 | — | 2 | 2 |
| 22.1-23.0 | 4 | 4 | 8 |
| 23.1-24.0 | 1 | — | 1 |
| 24.1-25.0 | — | 1 | 1 |
| 25.1-25.0 | 1 | — | 1 |
| 26.1-30.0 | 1 | 1 | 2 |
| Total | 31 | 20 | 51 |

TABLE 7
Blood glucose level after administration (mmol/L)

| Decrease in blood glucose level | Male | Female | Total |
|---|---|---|---|
| Ineffective | 1 | — | 1 |
| <1.0 | 1 | — | 1 |
| 1.1-2.0 | — | 1 | 1 |
| 2.1-3.0 | — | — | — |
| 3.1-4.0 | 1 | 2 | 3 |
| 4.1-5.0 | — | 1 | 1 |
| 5.1-6.0 | 1 | 1 | 2 |
| 6.1-7.0 | — | — | — |
| 7.1-8.0 | 4 | 1 | 5 |
| 8.1-9.0 | — | — | — |
| 9.1-10.0 | 6 | 1 | 7 |
| 10.1-11.0 | 5 | 3 | 8 |
| 11.1-12.0 | 6 | 3 | 9 |
| 12.1-13.0 | 4 | 3 | 4 |
| 13.1-14.0 | 3 | 2 | 5 |
| 14.1-15.0 | — | — | — |
| 15.1-16.0 | — | 1 | 1 |
| 16.1-17.0 | 1 | — | 1 |
| 17.1-18.0 | — | — | — |
| >18.1 | 2 | — | 2 |
| Total | 31 | 20 | 51 |

As seen from Tables 6 and 7, 49 out of 51 patients (96.08%) experienced decrease in blood glucose level by 3.1 mmol/L or more.

TABLE 8
Percentage of decrease in blood glucose level

| | Decrease (%) | | | | | | | |
|---|---|---|---|---|---|---|---|---|
| | Slight | 10-29 | 30-40 | 41-50 | 51-60 | 61-70 | 71-80 | >81 |
| No. of | 1 | 2 | 4 | 4 | 12 | 17 | 9 | 2 |
| cases | | | | | | | | |

TABLE 9
Days until decrease in blood glucose level
(days required to reach WHO standard)

| | Days | | | | |
|---|---|---|---|---|---|
| | 7 days | 15 days | 30 days | 45 days | 60 days |
| No. of cases | 14 | 11 | 19 | 6 | 1 |

Further, most of the subjects reported disappearance of the typical symptoms of diabetes, i.e. thirst, polyphagia and polyuria, and, in particular, significantly improved helplessness. The test substance according to the present invention was confirmed to have a distinct therapeutic effect on type 2 diabetes (blood sugar-lowering effect) and improving the typical diabetic symptoms.

[Health Food Composition Preparation Example 1] Drink

The composition for treating and preventing diabetes or diabetic complications (100 mg) was mixed with oligosaccharides syrup (25 g) and added to purified water (200 mL). The resulting solution was filled in plastic bags, 80 mL in each, and then sterilized at 130° C. for 4 to 5 seconds.

[Pharmaceutical Composition Preparation Example 1] Tablet

The composition for treating and preventing diabetes or diabetic complications (100 mg), cornstarch (100 mg), lactose (50 mg) and magnesium stearate (2 mg) were mixed and then prepared into a tablet according to a commonly employed method.

[Pharmaceutical Composition Preparation Example 2] Capsule

The composition for treating and preventing diabetes or diabetic complications (100 mg), crystalline cellulose (3 mg), lactose (14.8 mg) and magnesium stearate (0.2 mg) were mixed and then filled in a gelatin capsule according to a commonly employed method.

While the present invention has been described with respect to the specific embodiments, it will be apparent to those skilled in the art that various changes and modifications may be made without departing from the spirit and scope of the invention as defined in the following claims.

What is claimed is:

1. A composition for treating and preventing diabetes or diabetic complications comprising an extract of herbal combination of an ethanol extract of bitter melon, a caterpillar fungus extract and a hot-water extract comprising wolfberry tree bark (1): white mulberry bark (1-10): winged euonymus (1-10): kudzu root (1-10): Solomon's seal (1-10): white atractylodes rhizome (1-5): mondo grass rhizome (1-5): Japanese cornel fruit (1-5): ginseng (1-5), based on weight, as well as a fermented extract of bush clover as an effective ingredient.

2. The composition for treating and preventing diabetes or diabetic complications according to claim 1, wherein the extract of herbal combination comprises the ethanol extract of bitter melon, the caterpillar fungus extract and the hot-water extract at a mixing proportion of 4:1-4:2-5, based on weight.

3. The composition for treating and preventing diabetes or diabetic complications according to claim 1, wherein the extract of herbal combination is comprised in an amount of 0.1-50 wt % based on the total weight of the composition.

4. The composition for treating and preventing diabetes or diabetic complications according to claim 2, wherein the bitter melon extract is an ethanol extract obtained as powder after drying under reduced pressure.

5. The composition for treating and preventing diabetes or diabetic complications according to claim 1, wherein the fermented extract of bush clover is prepared by inoculating 20-40 wt % of a bush clover extract with Aspergillus oryzae and performing solid-state fermentation.

6. The composition for treating and preventing diabetes or diabetic complications according to claim 1, wherein the fermented extract of bush clover is comprised in an amount of 0.01-20 wt % based on the total weight of the composition.

7. The composition for treating and preventing diabetes or diabetic complications according to claim 5, wherein the bush clover extract is obtained by extracting bush clover with water, ethanol or a mixture solvent and then sterilizing at high temperature above the boiling point of ethanol (78.3° C.).

8. The composition for treating and preventing diabetes or diabetic complications according to claim 1, wherein the diabetes is type 1 diabetes or type 2 diabetes.

9. The composition for treating and preventing diabetes or diabetic complications according to claim 1, wherein the diabetic complication is neuropathy, diabetic retinopathy, nephropathy, sexual dysfunction, skin disease, hypertension, arteriosclerosis, stroke, heart disease or gangrene.
